(19) United States
(12) Patent Application Publication
(10) Pub. No.: US 2009/0306773 A1
(43) Pub. Date: Dec. 10, 2009

(54) OPAQUE CORNEAL INSERT FOR REFRACTIVE CORRECTION

(75) Inventors: Thomas Silversrini, Alamo, CA (US); Bruce A. Christie, Claremont, CA (US)

Correspondence Address: KNOBBE MARTENS OLSON & BEAR LLP, 2040 MAIN STREET, FOURTEENTH FLOOR, IRVINE, CA, 92614, US

(73) Assignee: AcuFocus, Inc., Irvine, CA (US)

(21) Appl. No.: 12/133,282

(22) Filed: Jun. 4, 2008

(51) Int. Cl. A61F 2/14; A61B 19/00

(52) U.S. Cl. 623/5.11; 623/5.12; 128/898

(57) ABSTRACT

Implants configured for corneal implantation are disclosed that have a light transmitting portion and a light-blocking portion disposed about the periphery of the implant. The implant can be an elongate member, such as a split or continuous ring, that can be implanted in the cornea to alter the refractive properties of the cornea by altering the curvature of the anterior surface of the cornea, thereby providing corrective refraction. The light-blocking portion can reduce edge effects, which may be visible or distracting to the patient.

### BACKGROUND OF THE INVENTION

1. Field of the Invention

This invention relates to methods and apparatuses for modifying the shape of portions of an eye, for example changing curvature of the cornea of the eye.

2. Description of the Related Art

Deviations from the proper shape of the corneal produce errors of refraction in the visual process. The properly shaped eye in a state of rest, without accommodation, focuses the image of distant objects exactly on the retina. Such an eye enjoys distinct vision for distant objects without effort. Any variation from this standard constitutes ametropia, a condition in which the eye at rest is unable to focus the image of a distant object on the retina. Hyperopia (“farsightedness”) is an error of refraction in which, with the eye at rest, parallel rays from distant objects are brought to focus behind the retina. Divergent rays from near objects are focused still further back.

The corneal surface of a hyperopic eye is often flattened, which decreases the angle of refraction of rays as they pass through the refractive surfaces of the cornea, causing a convergence or focus of the rays at a point behind the retina. The retina is the rear, internal surface of the eye that senses the light rays, which is later interpreted by the brain as an image. If light converges behind the retina, the image will appear blurred to the brain. Increasing the convexity of the hyperopic eye is one way of focusing rays of the hyperopic eye on the retina. Alternatively, a convex lens (such as a negative lens in a pair of eyeglasses or a negative contact lens) of sufficient strength to focus rays on the retina can be placed before the eye.

Myopia (“nearsightedness”) is a refractive condition in which parallel rays are brought to focus in front of the retina. One condition that commonly causes myopia is when the corneal curvature is steepened, in which the rays are refracted and converge or focus in front of the retina in the vitreous of the eye. When the front-to-back distance of the eyeball is too long, the rays reaching the retina are divergent, thus forming a circle of diffusion and a blurred image. A concave (positive) lens is often used in eyeglasses or contacts lenses to correct the focus of the eye for myopia.

Astigmatism is a condition that occurs when parallel rays of light do not focus to a single point within the eye, but rather have a variable focus due to the fact that the cornea is more curved in one meridian than in another. In this configuration, the eye refracts light rays in different meridians at different distances. Some degree of astigmatism is normal, but where it is pronounced, the astigmatism may require correction.

The normal treatment of these classic forms of refractive error of the eye is with the use of eyeglasses or contact lenses, as mentioned above. Unfortunately, eyeglasses and contact lenses have to be cleaned and periodically replaced. Eyeglasses may be especially cumbersome to carry and may be cosmetically unappealing. Contact lenses have to be replaced frequently and, consequently, can be quite expensive. Surgical procedures, such as laser corneal ablation, can be used to correct the curvature of the eye. These procedures are relatively complex and are often irreversible.

Another method for correcting eye disorders, such as myopia, is through the implantation of a ring in the eye's corneal stroma to change the curvature of the cornea. Such a ring can be inserted into a channel previously dissected in the stromal layer of the cornea. These rings are positioned within the stroma and outside of a projection of the eye's iris as viewed from the front of the eye. Unfortunately, these rings may have relatively large dimensions (e.g., inner diameters, outer diameters, and thicknesses). Large dimensions are required because these devices were placed outside of an optical zone of the eye. If placed close to the optical zone, these rings could cause optical distortions. Although placing these devices far from the optical zone reduces the likelihood of distortion, such peripheral placement forced these rings to be relatively thick so that the corneal curvature change induced at the periphery of the forward portion of the eye would induce sufficient change in curvature at the optical zone to provide a clinical benefit. These rings can have long healing times, in part due to their size, and may result in scar tissue build up, thus reducing the efficacy of the implanted rings.

### SUMMARY OF THE INVENTIONS

In some embodiments, an intracorneal implant that has an anterior surface and a posterior surface is configured for insertion into a cornea of a human eye. The implant comprises an elongated member comprising a nontransmissive, e.g., an opaque, portion extending along at least a portion of a periphery of the arcuate member. The elongate member can be arcuate in some cases. The implant is configured to alter the refractive properties of the cornea by altering the curvature of the anterior surface of the cornea.

In some variations, the intracorneal implant is dimensioned for placement in the cornea between the anterior surface and the posterior surface so that the implant surrounds an optical axis of the eye. The implant can alter the curvature of the cornea so as to provide a correction of between about 1.0 and about 3.0 diopters, in some cases between about 2.0 and about 5.0 diopters. In some cases, the implant can alter curvature to provide a correction of between about 2.0 and about 8.0 and in some cases between about 3.0 and about 10.0 diopters. The implant can alter curvature to provide a correction of between about 4.0 and about 15.0 diopters. In some variations, the implant can provide a correction of at least about 0.5 diopters.

When the opaque portion is exposed to incident light, the opaque portion blocks more than half of the incident light from passing therethrough. In one variation, the opaque portion has opacity of approximately 50% or more. In another variation, the opaque portion has opacity of approximately 60% or more. In another variation, the opaque portion has opacity of approximately 70% or more. The opaque portion can in some cases have opacity of approximately 80% or more. In another variation, the opaque portion has opacity of approximately 90% or more. In some variations, the opacity of the opaque portion can be less than the foregoing ranges. In some variations, for example, the opaque portion opacity is between about 85% and about 100%.

The elongated arcuate member generally defines an inner diameter equal to or greater than about 2.0 mm. In one variation, the elongated arcuate member defines an inner diameter of between about 2.0 mm and about 4.0 mm. In some variations, the elongated arcuate member defines an outer diameter equal to or less than about 4.5 mm. In one variation, the elongated arcuate member defines an outer diameter of at least about 2.5 mm. In some variations, the elongated arcuate member defines an outer diameter of between about 2.5 mm and about 4.5 mm. In some variations, the elongated arcuate member comprises an outer edge and an inner edge, wherein the outer edge defines an outer diameter in the range of about 2.5 mm to about 4.5 mm, and the inner edge defines an inner diameter in the range of about 2.0 mm to about 4.0 mm.

The elongated arcuate member has the inner edge that at least partially forms a generally circular opening. The elongated arcuate member preferably forms a split ring having the circular opening. In some embodiments, the implant comprises a lens that can be substantially clear and the elongated arcuate member forms at least a portion of a periphery of the lens. In some variations, the elongated arcuate member forms an annulus surrounding the lens.

The elongated arcuate member can comprise a split ring, as mentioned above, or a plurality of segmented members configured to form a substantially arcuate implant when implanted. In some embodiments, the plurality of segmented members can form a generally circular corneal implant. Each of the segmented members can be an arcuate member comprising opaque material.

In some embodiments, an intracorneal implant is configured for insertion into a cornea of a human eye having an anterior surface and a posterior surface. The implant comprises one or more light blocking portions. In some variations, the intracorneal implant comprises an elongated member having at least one light blocking portion extending along at least a portion of a periphery of the elongated member. In some variations, the implant is configured to alter the refractive properties of the cornea by altering the curvature of the anterior surface of the cornea. In some variations, the elongated member comprises one or more arcuate members. In one embodiment, the elongated member can subtend an angle of about 360 degrees. In another embodiment, the elongated member can subtend an angle equal to or less than about 270 degrees. In one embodiment, the elongated member can subtend an angle equal to or less than about 180 degrees. In one embodiment, the elongated member can subtend an angle equal to or less than about 90 degrees. In one embodiment, the elongated member can subtend an angle equal to or less than about 60 degrees. In one embodiment, the elongated member can subtend an angle equal to or less than about 30 degrees. In some variations, one or more of the arcuate members forming the elongated member can subtend at least one of the foregoing angles.

In some embodiments, a method for affecting the shape of a cornea of an eye is provided. The method comprises separating a first corneal layer and a second corneal layer. The anterior surface of the cornea can be altered to affect the refractive properties thereof by placing an opaque member between the first and second corneal layers.

In some variations, the opaque member comprises a first arcuate segment and a second arcuate segment. The altering of the anterior surface can comprise placing the first arcuate segment between the first and second corneal layers on one side of a central portion of the pupil and placing the second arcuate segment between the first and second corneal layers such that the central portion of the pupil is generally between the first and second arcuate segments.

In some variations, to implant the opaque member, an incision is formed in the anterior surface of the cornea. A channel is formed between the first and second corneal layers. The channel can be elongate in some techniques. In some techniques, the channel can be arcuate. The first end, the arcuate segment, and the second end are inserted through the incision. Any number of opaque members can be implanted using this technique.

In some variations, a first normal projection of the opaque member onto a plane perpendicular to an axis that extends through the pupil (e.g., through a center of the pupil and through a geometric center of the eye) is within a second normal projection of an inner edge of the iris of the eye onto the plane when the iris is dilated. The iris can be partially or fully dilated.

In some embodiments, the outer periphery of the opaque member defines a first dimension and an inner edge of an iris of the eye defines a second dimension when the pupil is dilated (either fully or partially dilated). The first dimension is equal to or less than the second dimension. At least a portion of the outer periphery can be positioned within the optical zone of the eye.

### BRIEF DESCRIPTION OF THE DRAWINGS

The above-mentioned and other features of the invention disclosed herein are described below with reference to the drawings of preferred embodiments. The illustrated embodiments are intended to illustrate, but not to limit the invention. The drawings comprise the following figures.

FIG. 1 is a schematic representation of a horizontal section of the eye.

FIG. 2A is a schematic illustration of the anterior portion of the eye showing the various layers of the cornea. The iris of the eye is in a constricted position.

FIG. 2B is a schematic illustration of the anterior portion of the eye of FIG. 2A showing the iris of the eye in a dilated position.

FIG. 2C is a schematic illustration showing relative positions of an intracorneal ring (“ICR”) and an inner edge of an iris based upon projections of these structures onto a plane perpendicular to an axis intersecting the cornea.

FIG. 3 is a schematic representation showing how light focuses on the retina of a normal eye.

FIG. 4 is a schematic representation of how light does not focus on the retina of a myopic eye.

FIG. 5 is a perspective view of an annular intracorneal implant that is positioned within a cornea of an eye.

FIG. 6A is a top plan view of an intracorneal implant in accordance with one embodiment.

FIG. 6B is a top plan view of an intracorneal implant in accordance with another embodiment.

FIG. 7 is a cross-sectional view of the intracorneal implant of FIG. 6A taken along the line 7-7.

FIG. 8 is a top plan view of a segmented implant that can be positioned in a cornea of an eye.

FIG. 9A is a top plan view of a segmented implant in accordance with another embodiment.

FIG. 9B is a top plan view of a two segment segmented implant applied to an eye.

FIG. 9C is a graphical illustration of the curvature change in first and second planes compared to an astigmatic condition.

FIGS. 10-14 are cross-sectional views of portions of intracorneal implants.

FIGS. 15-18 are cross-sectional views of portions of intracorneal implants having light blocking layers.

FIG. 19 is a cross-sectional view of a disk-shaped corneal insert for implantation in an eye.

FIG. 20 is a top plan view of the corneal insert of FIG. 19.

FIG. 21 is a schematic representation of initial steps of a technique for accessing internal corneal layers.

FIG. 22 illustrates a flap of a dissected cornea positioned to expose an interior portion of the cornea.

FIG. 23 illustrates the disk-shaped corneal insert of FIGS. 19 and 20 implanted in the interior portion of the cornea while the flap is pulled back.

FIG. 24 illustrates the flap overlaying the corneal insert.

### DETAILED DESCRIPTION OF THE PREFERRED EMBODIMENTS

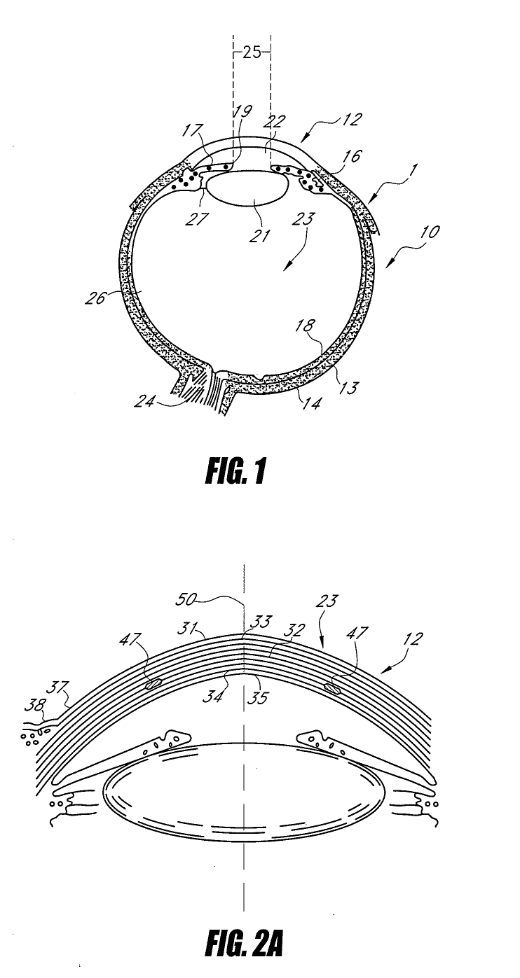

FIG. 1 shows a horizontal section of an eye 1, which has a generally spherical portion, referred to herein as an eyeball 10. The eye 1 includes a cornea 12, which is an anterior bulged spherical portion of the eye 1, and a sclera 13 enclosing the transparent media through which the light passes to reach the retina 18. The retina 18 includes light sensitive tissue and is located at the back of the eye 1. The sclera 13 is a fibrous protective portion and constitutes approximately the posterior ⅚ of the eyeball 10. The sclera 13 is white and opaque and the visible portion of the sclera is sometimes referred to as the “white” of the eye. The anterior ⅙ of the eyeball 10 is the cornea 12.

An interior covering or layer of the eye 1 is vascular and nutritive in function and includes the choroid 14, the ciliary body 16, and the iris 17. This interior covering maintains the retina 18. The ciliary body 16 supports a lens 21 and is involved in accommodation. The iris 17 is the most anterior portion of the interior covering of the eye 1 and is arranged in a frontal plane. The iris 17 is a thin circular disc and is perforated near its center by a circular aperture called the pupil 19. The iris 17 is analogous to the diaphragm of a camera in that the iris varies the size of the pupil 19 to regulate the amount of light that reaches the retina 18. The iris 17 divides the space between the cornea 12 and a lens 21 into an anterior chamber 22 and posterior chamber 23. The retina 18, which consists of nerve elements, is a further internal layer covering disposed over the choroid 14. The nerve elements form the true receptive, light sensing portion of the eye 1 for capturing visual impressions.

A layer of special visual cells or photoreceptors called rods and cones lie just beneath a pigmented epithelium on the anterior wall of the retina 18. These cells transform physical energy in the form of light into nerve impulses transmitted along the optic nerve 24.

A vitreous body 26 resides between the lens 21 and the retina 18. The vitreous body 26 is a transparent gelatinous mass, which fills the posterior ⅘ of the eyeball 10. The ciliary body 16 and the retina 18 are supported about the periphery of the vitreous body 26. A frontal saucer-shaped depression in the vitreous body 26 abuts and supports a posterior portion of the lens 21. The lens 21 of the eye 1 is a transparent bi-convex body of crystalline appearance placed between the iris 17 and vitreous body 26. Its axial diameter varies with accommodation. It is the deformation of the lens 21 that enables the eye 1 to cause light rays from objects that are located at a range of distances from the eye to converge on the retina 18.

The cornea 12 is a fibrous portion of the eyeball 10 that is highly light transmissive. Sometimes the cornea 12 is more curved in one meridian than another giving rise to astigmatism. Astigmatism, like myopia and hyperopia, discussed above, is a refractive error of the eye that can be treated by optic devices described herein. A central portion of the cornea, e.g., a central approximate one-third, is the portion through which light entering the eye can be transmitted to the retina and is therefore sometimes called the “optical zone” 25. Most of the refraction of the eye 1 takes place through the cornea 12.

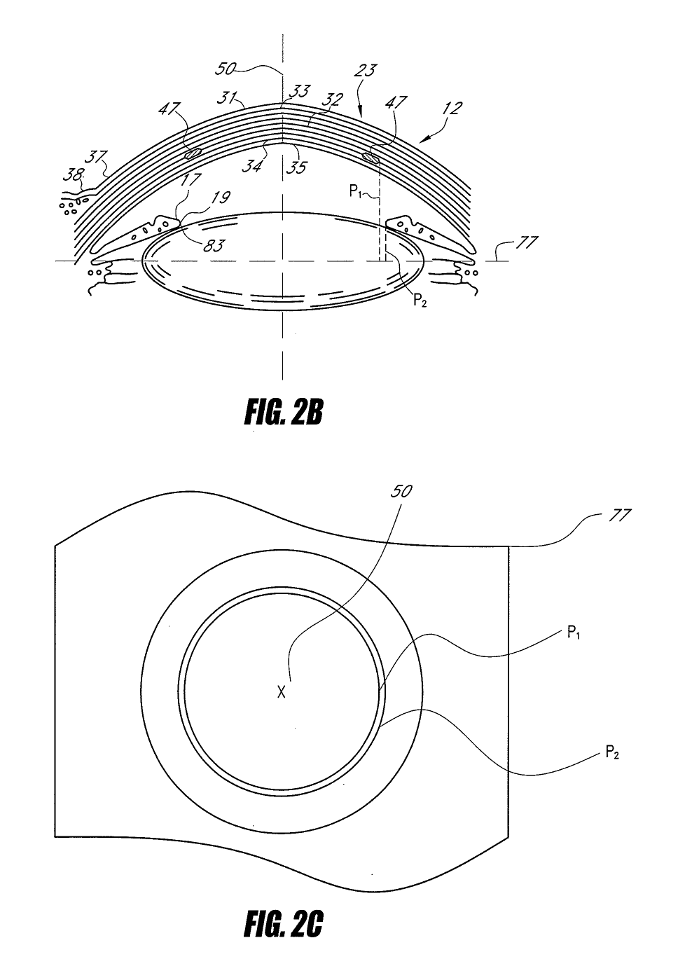

FIGS. 2A and 2B show a more detailed drawing of an anterior portion of the eyeball 10 that shows different layers of the cornea 12, including an outer layer called the epithelium 31 and an internal layer called the stroma 32. The epithelium 31 includes a thin layer of cells that act as a protective layer of the cornea 12. These epithelial cells are rich in glycogen, enzymes and acetylcholine and their activity regulates the corneal corpuscles and controls the transport of water and electrolytes through more posterior layers of the cornea 12, such as through lamellae of the stroma 32.

An anterior limiting lamina 33, referred to as Bowman's membrane, is located between the epithelium 31 and the stroma 32. The stroma 32 is comprised of lamella or layers having bands of fibrils parallel to each other and crossing the whole of the cornea 12. While most of the fibrous bands are parallel to the surface of the cornea 12, some are oblique, especially anteriorly. A membrane called the “Descemet's membrane” 34 forms a posterior limiting lamina and is a strong membrane sharply defined from the stroma 32.

The cornea 12 also includes posterior-most layer called the endothelium 35 that consists of a single layer of cells that aid in maintaining the transparency of the cornea. The eyeball 10 also includes a limbus 37 and conjunctiva 38. The limbus 37 is a transition zone between the conjunctiva 38 and sclera 13 and the cornea 12. FIGS. 2A and 2B show an intracorneal ring (“ICR”) 47 that is capable of altering the shape of the cornea to compensate for refractive error of the eye 1 implanted in the stroma 32 of the cornea 12. Although shown as a ring, the “ICR” can take other shapes and can more generally be described as an ocular device capable of altering corneal curvature to compensate for refractive error.

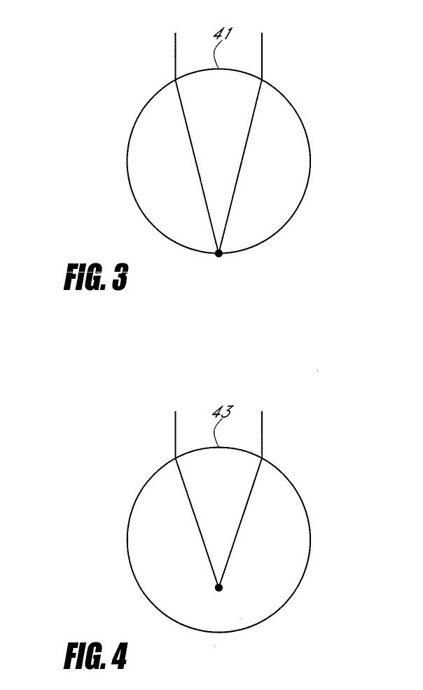

FIG. 3 shows the globe of a properly functioning eye, having a cornea 12 with a normal curvature 41. If parallel rays of light pass through the corneal surface of FIG. 3, they are refracted by the corneal surfaces and converge eventually on or very near the retina of the eye. FIG. 3 schematically illustrates the overall refractive effect of the eye, including the lens of the eye. The eye depicted in FIG. 4 is myopic, with light rays refracted into focus at a point in the vitreous body, forward of the retinal surface. If an intracorneal ring is implanted into the cornea such that the radius of curvature of the cornea is increased, e.g., uniformly increased, the central portion of the cornea is generally flattened. Light rays refracted by the now flattened corneal surface will be refracted at a smaller angle and thus converge at a more distant point, such as directly on the retina.

The intracorneal rings disclosed herein can be utilized to adjust the curvature of the cornea 12. By varying parameters of the intracorneal ring, such as its thickness, shape and circumference, the shape of the cornea can be changed in a variety of ways. Where there is serious astigmatism, the natural asphericity will not be altered such that the astigmatism will be significantly increased. However, where there is significant astigmatism that results in impaired vision, the intracorneal ring may actually improve the asphericity to reduce such astigmatism and improve vision.

With reference again to FIG. 2A, an intracorneal ring (ICR) 47 is shown implanted in the stromal layer of the cornea 12 to improve the refractive properties of the cornea. The illustrated ICR 47 alters the refractive properties of the cornea 12 by altering the curvature of the anterior surface 31 of the cornea 12. The curvature of the anterior surface 31 of the cornea 12 can be changed for a corrective refraction of at least 1.0 diopter. In some embodiments, the geometry of the ICR 47 and location of implantation can be selected to achieve a corrective refraction of between about 1.0 and 3.0 diopters. In some embodiments, the geometry of the ICR 47 and location of implantation can be selected to achieve a corrective refraction of between about 2.0 and 5.0 diopters. In some variations, the geometry of the ICR 47 and location of implantation can be selected to achieve a corrective refraction of between 2.0 and 8.0 diopters. In some variations, the geometry of the ICR 47 and location of implantation can be selected to achieve a corrective refraction of between 3.0 and 10.0 diopters. In some variations, the geometry of the ICR 47 and location of implantation can be selected to achieve a corrective refraction of between about 4.0 and 15.0 diopters. The amount of corrective refraction for the various thicknesses and diameters of ICRs of different cross-sectional shaped rings may differ from those values depending on the individual patient and variety of factors as are known in the art.

By selecting the thickness of the ICR 47 according to the amount of correction necessary, the rays refracted by the cornea 12 and other eye components can be brought to focus directly on the retina 18 as desired. The ICR 47 can be relatively thin but can still produce a relatively large corneal reflective corrective change because the ICR 47 is implanted in or closer to the optical zone of the cornea than prior devices.

The ICR 47 can be positioned based on the positions of the iris 17 in a dilated and constricted position. As mentioned above, the iris 17 constricts and dilates to regulate the amount of light reaching the retina 18. When the iris 17 is fully constricted (FIG. 2A), the average human iris 17 has a diameter of about 1.5 mm. The constricted iris can be between about 1 and 2 mm. The iris 17 can dilate from the constricted position to a dilated position as the ambient light intensity is reduced. FIG. 2B shows the iris 17 in a dilated position. In relatively low light conditions, the dilated iris 17 can have a diameter of about 8 mm in dark conditions. Of course, the dimensions of the iris 17 can vary between patients. The illustrated ICR 47 is positioned within the dilated iris 17 as viewed from the front of the eye. More particularly, the ICR 47 can be positioned within a projection of the iris 17 in some embodiments and in some applications. The outer dimension of the ICR 47 can be equal to or smaller than the diameter of the pupil 19 at least when the iris 17 is partially or fully dilated.

As best seen in FIGS. 2B and 2C, a first projection P1 of the ICR 47 onto a plane 77, which is preferably perpendicular to an axis 50 extending through a central portion of the pupil 19 (e.g., through the geometric center of the pupil or the intersection of the line of sight), is near to or within a second projection P2 of an inner edge of the iris 17 of the eye onto the plane 77 when the iris 17 is partially or fully dilated. In some embodiments, the first projection P1 of at least a portion of the ICR 47 onto the plane 77 is spaced from and within the second projection P2 of the inner edge of the iris 17 onto the plane 77 when the iris 17 is fully dialed. Preferably, the entire projection of the ICR 47 is within the second projection P2 of the inner edge of the iris 17. The ICR 47, however, can also be implanted at other suitable locations.

At least a portion of the ICR 47 can be light blocking to minimize, reduce, or substantially eliminate one or more types of optical distortions. As used herein, the term “optical distortion” is a broad term and includes, without limitation, scattering of light, edge effects (e.g., edge glare), or other optical disorder conditions that result in distortions or blurring of the image on the retina 18. For example, if a traditional corneal ring were implanted within the optical zone 23, light rays passing through the cornea would cause optical distortions (e.g., edge effects), thus producing an improperly focused image. Various types of optical distortions can result in light rays not properly converging on the retina 18, thereby reducing the quality of the observed image. Advantageously, the light blocking portions can be strategically positioned throughout the ICR 47 so as to diminish, reduce, or completely eliminate one or more of these types of optical distortions that would otherwise be produced if the ICR comprised a completely transparent material.

The implanted ICR 47 preferably surrounds the axis 50 extending through the eye. In some embodiments, the axis 50 is an optical axis extending through a center section of the pupil 19. For example, the axis 50 can generally coincide with the center of the pupil 19. Additionally or alternatively, the axis 50 can be an axis extending through the geometric center of the eye. The optical axis 50 can extend generally through the center of the cornea 12, the lens 21, and intersect a central portion of the retina 18. In some embodiments, the optical axis 50 extends along the line of sight of the eye. The implanted ICR 47 preferably surrounds the axis 50 and, in some embodiments, is generally centered with respect to the axis 50. In one arrangement, the center of the cornea 12 can be used as a reference point for implantation of the ICR 47. The ICR 47 can be implanted at other locations as well.

With continued reference to FIGS. 2A and 2B, ambient light passes through the modified cornea 12 and is preferably properly refracted and generally focused on the retina 18. The implanted ICR 47 can be within the optical zone 25 in at least some conditions within the group of rays of light that ultimately reach the retina 18. When the iris 17 is dilated, light can pass through the cornea 21 past the ICR 47 and through the pupil 19 to the retina 18. Depending on its dimensions and configuration, the ICR 47 may be visible to the patient. Though located in the optical zone 25, the ICR 47 may be visually negligible because a person may become accustomed to the ICR 47 over time. For example, the person may subconsciously ignore or filter out a light absorbing or dark edge of the ICR 47, and consequently, the ICR 47 may not significantly degrade the viewed image. In other embodiments, the ICR 47 may not be visually noticeable to the patient.

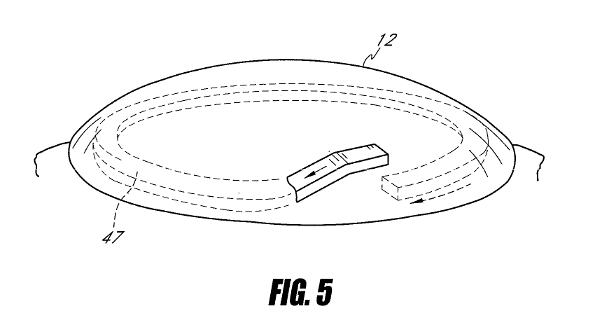

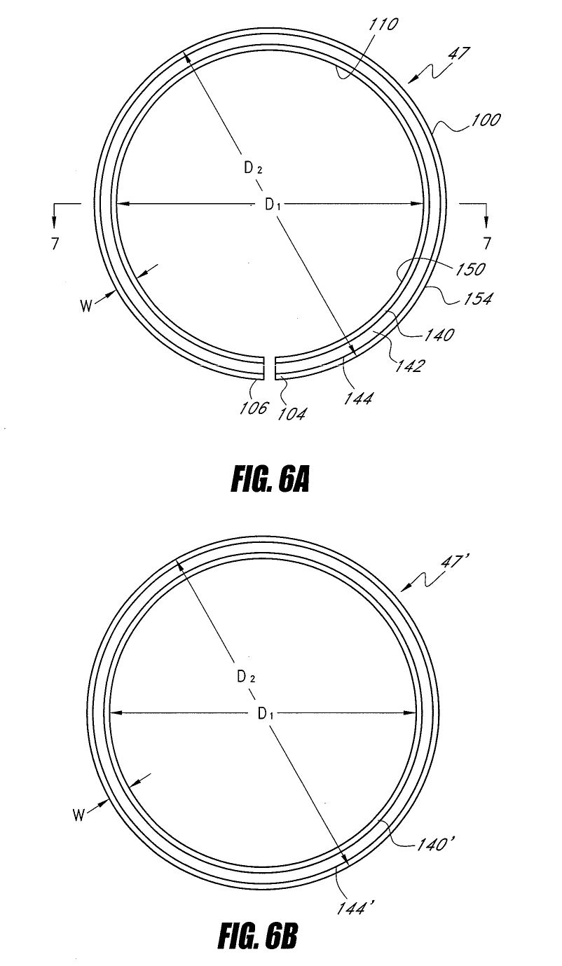

FIG. 5 is a perspective view of the ICR 47 being inserted within the cornea 12. In the embodiment shown, the ICR 47 is oblong in cross-section. In the use of one oblong configuration, the ICR 47 is placed such that the widest edge of the cross-section of the ICR 47 does not reside in a plane that is perpendicular to the diametral axis of the ICR. In particular, FIGS. 6A and 6B illustrate an ICR 47 that is generally trapezoidal in cross-section. In one embodiment, the widest side of the cross-section in this variation of the ICR 47 is disposed along a line that defines an angle of from about forty-five to ninety degrees to a line intersecting the mid-point of a diameter of the ICR, which can correspond to the line-of-sight of the eye when the ICR is applied. In one application of an oblong ICR 47, the narrower profile or edge of the cross-section faces radially inward toward the line of sight. The wide and narrow edges can but need not be perpendicular to each other.

The ICR 47 can be inserted into the corneal stroma of the eye through an incision. The ICR 47 can be inserted through the incision between adjacent layers of the corneal stroma. In some embodiments, the incision can be a 2.5 mm incision placed peripherally into the corneal stroma. In general, the size of the incision can be between approximately 0.5 mm and approximately 5.0 mm in length, e.g., approximately 2.5 mm in length.

Prior to inserting the ICR 47, a blade (e.g., a channeling or pocketing blade) is inserted at the depth of the incision and a circular channel is cut into the corneal stroma. Proper centering of the cut is accomplished by use of a centering device that aligns the channeling blade. The ICR 47 is then inserted and advanced through the channel. In some embodiments, the ends of the ICR 47 are secured to each other by the overlapping end portions, which may be supplemented by fastening one end to the other. However, such fastening of the end portions is not required. U.S. Pat. No. 5,323,788, which is incorporated by reference, discloses systems, devices, materials, methods (e.g., methods of implantation) and techniques that can be used alone or in combination with present disclosure. As detailed below, various other techniques can be used to implant the ICR 47.

FIG. 6A is a top elevational view of an ICR 47 in the form of a split ring illustrated in an implanted configuration. The ICR 47 comprises an elongated member 100 that extends between two opposing ends 104, 106. When placed in the cornea 12, the elongated member 100 can fill a generally annular space or can elevate a generally annularly shaped portion of corneal tissue. An aperture or opening 110 can be defined by the inner periphery 140 of the ICR 47. As detailed below, the size and configuration of the elongated member 100 can be selected to achieve the desired refractive correction by effectively altering the curvature of the cornea 12.

The split ICR 47 can be generally circular as viewed from above, as shown in FIG. 6A. The elongated member 100 can also be somewhat elliptical, polygonal, or have other non-circular configurations. The illustrated ICR 47 is a circular annulus wherein the elongated member 100 defines an unbroken arc extending between the adjacent ends 104, 106. In some embodiments, the ends 104, 106 couple together when in the implanted configuration. U.S. Pat. No. 5,323,788 discloses various types of ends that can be used for interlocking ends that can be employed.

At least a portion of the ICR 47 can be light-blocking as discussed above in connection with FIGS. 2A and 2B. The light blocking portion can reduce, limit, or substantially eliminate optical distortions, such as edge effects. For example, the light blocking portions can be positioned at the outer periphery or edges of the ICR 47. The light blocking portion can comprise an opaque material or other material suitable for effectively blocking the passage of light through the ICR 47. As used herein, the term “opaque” is a broad term and means, without limitation, not transmitting light or radiant energy. In some embodiments, the opaque material can prevent transmission of light by absorption of light incident thereon. In some embodiments, the opaque material can prevent transmission of light by scattering light incident thereon. The ICR 47 can comprise opaque materials with different transmissive properties. In some embodiments, the ICR 47 comprises an opaque material that has opacity of about 25%. That is, when the ICR 47 is exposed to light, the opaque material blocks about 25% of the light, thus allowing about 75% of the light to pass therethrough. In some embodiments, the ICR 47 comprises an opaque material having opacity of at least 30%. The ICR 47 comprises an opaque material having opacity of at least 40% in one embodiment and in another embodiment opacity of at least 50%. The ICR 47 can comprise an opaque material having opacity of 60% or more and in some embodiments, at least 70%. In one variation, the ICR 47 comprises an opaque material having opacity of at least 75%, while in another variation the ICR 47 has an opaque material having opacity of at least 80%. In one variation, the ICR 47 comprises an opaque material having opacity of at least 90%. In some embodiments, the ICR 47 at least partially or completely comprises an opaque material that blocks more than three-quarters of the incident light from passing therethrough. In some embodiments, the ICR 47 comprises an opaque material that, when exposed to incident light, blocks a substantial portion of the incident light from passing therethrough. The opaque material can be selected based on the ring's geometry, mechanical properties, optical properties (including opacity or transmissivity), and/or biocompatibility.

Opacity of the ICR may be achieved in any of several different ways. For example, in one embodiment, the material used to make ICR may be naturally opaque. Alternatively, the material used to make the ICR may be substantially clear, but treated to produce a desired degree of opacity, such as those listed above. One or more dyes, pigmentation agents, or other additives can be combined with the clear material to render the material substantially or completely opaque, for example. The amount of the additive can be selected to achieve the desired degree of opacity. In still another example, the surface of the ICR may be treated physically or chemically (such as by etching) to alter the refractive and transmissive properties of the ICR and make it less transmissive to light.

In still another alternative embodiment, the surface of the ICR may be coated with a material to make the ICR less transmissive to light. For example, the surface of the ICR may be coated with metal, titanium, gold, carbon, or other suitable material to provide opacity to the surface of the ICR. Various types of coating (e.g., plating and deposition) techniques can be used to form a coating that increases the overall opacity of the ICR. Additionally, the ICR may be patterned to provide areas of varying light transmissivity. In particular, it may be advantageous to modify the edge or boundary of the nontransmissive region to further reduce any edge effects that the patient might notice. For example, it may be advantageous to soften the edge or boundary. Any of a number of techniques can be employed in this regard, which techniques are sometimes referred to as apodization, including providing a gradual change in opacity from a nearly completely opaque at a location spaced from the edge to fifty percent transmission or more near the edge. A variety of other apodization techniques are set forth in U.S. Pat. Nos. 5,662,706; 5,905,561; and 5,965,330, which are all hereby incorporated by reference herein in their entireties.

Various types of fabrication processes can be used to make at least a portion of the ICR. Different coating processes, for example, can be used to deposit material on the surfaces of the ICR. Coating processes include, but are not limited to, sputtering, chemical vapor deposition (CVD), and other coating processes.

CVD methods and other analogous layer forming techniques may also be used to form the ICR or a portion of the ICR. CVD methods can include atmospheric pressure chemical vapor deposition, low-pressure chemical vapor deposition, plasma assisted (enhanced) chemical vapor deposition, photochemical vapor deposition, laser chemical vapor deposition, metal-organic chemical vapor deposition, and chemical beam epitaxy. Other suitable techniques for forming light blocking layers that can be used include, without limitation, electroplating or electrodepositing and electroforming. Some such techniques are described in U.S. application Ser. No. 11/107,359, entitled “Method of Making an Ocular Implant” filed Apr. 14, 2005, which is hereby incorporated by reference in its entirety.

In one embodiment, opaque portions of the ICR are formed by, for example, electroplating or electrodeposition processes. Electrodeposition is the process of producing a layer or coating, which can be metallic, on a surface of an object by the action of electric current. The deposition of a metallic coating onto an object can be achieved by negatively charging the object to be coated and immersing the object into a solution that contains a salt of the metal to be deposited. In this arrangement, the object to be plated can be the cathode of an electrolytic cell. In one technique, the object to be plated is similar to the substrate ICR. The substrate can be a conductive polymer, metal, or other suitable material.

In one technique, the metallic ions of the salt carry a positive charge and are attracted to the substrate or object. When the metallic ions reach the negatively charged object, e.g., a substrate, the substrate provides electrons to reduce the positively charged ions to metallic form. In electrodepositing and electroforming, the substrate can be made of any suitable material, e.g., a conductive polymer, copper, etc. The material to be plated is one that can be eroded to separate an ICR from the substrate. Where the material to be plated is intended to be a layer or a portion of the ICR, the material is selected with the biocompatility and stability properties discussed herein for long implantation life (e.g., the material can be opaque, inert, and preferably does not degrade in the presence of UV radiation). The substrate material of the ICR may be conductive, such as a conductive polymer that is coated with an opaque metallic material through an electroplating process.

The ICR 47 can comprise a light blocking portion that is formed separately from the underlying ring. The light blocking portion can be formed by a molding process (e.g., compression molding, injection molding, etc.), machining, stamping, or other suitable manufacturing process. In some embodiments, the light blocking portion can be formed through a machining process, such as cutting on a lathe. The starting material can be metal that is turned and cut on the lathe in the desired shape. The cut metal piece can then be assembled with a polymer underlying ring to form hybrid insert.

The ICR 47 preferably comprises one or more somewhat opaque biocompatible materials. The opaque materials can comprise, but are not limited to, polymers, elastomers, polyacrylates, silicones, isoprene, polycarbonates, polyolefins such as polyethylene, polypropylene, and polybutylene, their mixtures and polyolefin interpolymers, block copolymers and the like. An optically clear material can be altered to achieve the desired opaque characteristics, as mentioned above. Dies or other additives can be mixed with polymers to obtain the desired opacity. Alternatively or additionally, the ICR 47 can comprise one or more non-transmissive materials, such as metals, to form the ring. In some embodiments, the ICR 47 can comprise, but are not limited to, stainless steel, shape memory materials (e.g., nitinol), titanium (e.g., titanium 6 aluminum 4 vanadium), aluminum, TiNi and other alloys of TiNi, gold, tantalum, and platinum are believed to have these properties and to be able to perform well in ocular applications, as well as other materials suitable for use in the human body. These metals can be treated to achieve desired optical characteristics. For example, metals can be treated to turn them black or any other suitable color.

Another class of materials that may be advantageous in some applications includes materials that have superelastic or shape memory characteristics. Such materials include, without limitation, nitinol and other similar alloys of nickel and titanium. Such materials are particularly well suited for implantation using less invasive techniques, such as implantation in a pocket. These materials are advantageous in that they can enable an implant to be folded, rolled, or otherwise compressed to a relatively low profile so that the ICR can fit through a relatively small incision.

In some embodiments, an agent can be used to produce an opaque material, for example, at least a portion ICR can comprise at least one opacification agent which imparts a desired degree of opacity. In some embodiments, the opacification agent provides sufficient opacity to produce the depth of field improvements described herein, e.g., in combination with a transmissive aperture. In one embodiment, the opacification agent renders the material opaque. In another embodiment, the opacification agent prevents transmission of about 90 percent or more of incident light. In another embodiment, the opacification agent prevents transmission of about 80 percent or more of incident light. Preferred opacification agents include, but are not limited to organic dyes and/or pigments, preferably black dyes, such as azo dyes, hematoxylin black, and Sudan black inorganic dyes and/or pigments, including metal oxides such as iron oxide black and ilminite, silicon carbide and carbon (e.g. carbon black, a submicron powdered carbon). The foregoing materials may be used alone or in combination with one or more other materials disclosed herein. The opacification agent may be applied to one or more surfaces of the ICR on all or some of the surfaces, or it may be mixed or combined with the polymeric material (e.g. blended during the polymer melt phase). Although any of the foregoing materials may be used, carbon may be especially useful in that it does not fade over time as do many organic dyes, and that it also aids the UV stability of the material by absorbing UV radiation.

Some opacification agents, such pigments, which are added to blacken, darken or opacify portions of the ICR may cause the ICR to absorb incident radiation to a greater degree than ICR material not including such agents. Because the matrix polymer that carries or includes the pigments or particles in such embodiments may be subject to degradation from the absorbed radiation, it is preferred that the ICR, which may be vulnerable to environmental degradation, is made of a material which is itself resistant to degradation such as from UV radiation, or that it be generally transparent to or non-absorbing of UV radiation.

Also, in some embodiments, an ICR can have an appreciable surface to volume ratio or at least a significant amount of surface area exposed to a great deal of sunlight following implantation. In such embodiments, an ICR preferably comprises a material that has good resistance to degradation, including from exposure to ultraviolet (UV) or other wavelengths of light. Polymers including a UV absorbing component, including those comprising UV absorbing additives or made with UV absorbing monomers (including co-monomers), may be used in forming ICRs as disclosed herein which are resistant to degradation by UV radiation. Examples of such polymers include, but are not limited to, those described in U.S. Pat. Nos. 4,985,559 and 4,528,311, the disclosures of which are hereby incorporated by reference in their entireties. In one embodiment, the ICR comprises a material which itself is resistant to degradation by UV radiation. In one embodiment, the ICR comprises a polymeric material that is substantially reflective of or transparent to UV radiation.

Alternatively, the ICR may include a component which imparts a degradation resistive effect, or may be provided with a coating, preferably at least on the anterior surface, which imparts degradation resistance. Such components may be included, for example, by blending one or more degradation resistant polymers with one or more other polymers. Such blends may also comprise additives that provide desirable properties, such as UV absorbing materials. In one embodiment, blends preferably comprise a total of about 1-20 wt. %, including about 1-10 wt. %, 5-15 wt. %, and 10-20 wt. % of one or more degradation resistant polymers. In another embodiment, blends preferably comprise a total of about 80-100 wt. %, including about 80-90 wt. %, 85-95 wt. %, and 90-100 wt. % of one or more degradation resistant polymers. In another embodiment, the blend has more equivalent proportions of materials, comprising a total of about 40-60 wt. %, including about 50-60 wt. %, and 40-50 wt. % of one or more degradation resistant polymers. ICRs may also include blends of different types of degradation resistant polymers, including those blends comprising one or more generally UV transparent or reflective polymers with one or more polymers incorporating UV absorption additives or monomers. These blends include those having a total of about 1-20 wt. %, including about 1-10 wt. %, 5-15 wt. %, and 10-20 wt. % of one or more generally UV transparent polymers, a total of about 80-100 wt. %, including about 80-90 wt. %, 85-95 wt. %, and 90-100 wt. % of one or more generally UV transparent polymers, and a total of about 40-60 wt. %, including about 50-60 wt. %, and 40-50 wt. % of one or more generally UV transparent polymers. The polymer or polymer blend may be mixed with other materials as discussed below, including, but not limited to, opacification agents, polyanionic compounds and/or wound healing modulator compounds. When mixed with these other materials, the amount of polymer or polymer blend in the material which makes up the ICR is preferably about 50%-99% by weight, including about 60%-90% by weight, about 65-85% by weight, about 70-80% by weight, and about 90-99% by weight.

Some embodiments employ degradation resistant polymers include halogenated polymers. Preferred halogenated polymers include fluorinated polymers, that is, polymers having at least one carbon-fluorine bond, including highly fluorinated polymers. The term “highly fluorinated” as it is used herein, is a broad term used in its ordinary sense, and includes polymers having at least one carbon-fluorine bond (C—F bond) where the number of C—F bonds equals or exceeds the number of carbon-hydrogen bonds (C—H bonds). Highly fluorinated materials also include perfluorinated or fully fluorinated materials, materials that include other halogen substituents such as chlorine, and materials that include oxygen- or nitrogen-containing functional groups. For polymeric materials, the number of bonds may be counted by referring to the monomer(s) or repeating units that form the polymer, and in the case of a copolymer, by the relative amounts of each monomer (on a molar basis).

Preferred highly fluorinated polymers include, but are not limited to, polytetrafluoroethylene (PFTE or Teflon®), polyvinylidene fluoride (PVDF or Kynar®), poly-1,1,2-trifluoroethylene, and perfluoroalkoxyethylene (PFA). Other highly fluorinated polymers include, but are not limited to, homopolymers and copolymers including one or more of the following monomer units: tetrafluoroethylene —(CF₂—CF₂)—; vinylidene fluoride —(CF₂—CH₂)—; 1,1,2-trifluoroethylene —(CF₂—CHF)—; hexafluoropropene —(CF(CF₃)—CF₂)—; vinyl fluoride —(CH₂—CHF)— (homopolymer is not “highly fluorinated”); oxygen-containing monomers such as —(O—CF₂)—, —(O—CF₂—CF₂)—, —(O—CF(CF₃)—CF₂)—; chlorine-containing monomers such as —(CF₂—CFCl)—. Other fluorinated polymers, such as fluorinated polyimide and fluorinated acrylates, having sufficient degrees of fluorination are also contemplated as highly fluorinated polymers for use in corneal implants, e.g., ICRs and lenses, according to preferred embodiments. The homopolymers and copolymers described herein are available commercially and/or methods for their preparation from commercially available materials are widely published and known to those in the polymer arts.

Although highly fluorinated polymers are preferred, polymers having one or more carbon-fluorine bonds but not falling within the definition of “highly fluorinated” polymers as discussed above, may also be used. Such polymers include co-polymers formed from one or more of the monomers in the preceding paragraph with ethylene, vinyl fluoride or other monomer to form a polymeric material having a greater number of C—H bonds than C—F bonds. Other fluorinated polymers, such as fluorinated polyimide, may also be used. Other materials that could be used in some applications, alone or in combination with a fluorinated or a highly fluorinated polymer, are described in U.S. Pat. No. 4,985,559 and in U.S. Pat. No. 4,538,311, both of which are hereby incorporated by reference herein in their entirety.

The preceding definition of highly fluorinated can be illustrated by a few examples. One preferred UV-resistant polymeric material is polyvinylidene fluoride (PVDF), having a structure represented by the formula: —(CF₂—CH₂)ₙ—. Each repeating unit has two C—H bonds, and two C—F bonds. Because the number of C—F bonds equals or exceeds the number of C—H bonds, PVDF homopolymer is a “highly fluorinated” polymer. Another material is a tetrafluoroethylene/vinyl fluoride copolymer formed from these two monomers in a 2:1 molar ratio. Regardless of whether the copolymer formed is block, random or any other arrangement, from the 2:1 tetrafluoroethylene:vinyl fluoride composition one can presume a “repeating unit” comprising two tetrafluoroethylene units, each having four C—F bonds, and one vinyl fluoride unit having three C—H bonds and one C—F bond. The total bonds for two tetrafluoroethylenes and one vinyl fluoride are nine C—F bonds, and three C—H bonds. Because the number of C—F bonds equals or exceeds the number of C—H bonds, this copolymer is considered highly fluorinated.

Certain highly fluorinated polymers, such as PVDF, have one or more desirable characteristics, such as being relatively chemically inert and having a relatively high UV transparency as compared to their non-fluorinated or less highly fluorinated counterpart polymers. Although the applicant does not intend to be bound by theory, it is postulated that the electronegativity of fluorine may be responsible for many of the desirable properties of the materials having relatively large numbers of C—F bonds.

Highly fluorinated polymers, PVDF, and the other materials and classes of materials disclosed herein can be used alone or in combination with carbon black or other suitable opacification agents and other additives that provide advantageous features in an ICR. An ICR can include a polyanionic compounds like proteoglycans and glycosaminoglycans to provide advantageous features as discussed below. Additional polyanionic compounds and other additives of particular use include glucose 6 phosphate, dermatan sulfate, chondroitin sulfate, keratan sulfate, heparan sulfate, heparin, dextran sulfate, hyaluronic acid, pentosan polysulfate, xanthan, carrageenan, fibronectin, laminin, chondronectin, vitronectin, poly L-lysine salts, and alginate. In some embodiments, a useful additive includes dextran sulfate.

In addition, it may be useful to incorporate into an ICR a wound healing modulator, which can be loaded into the polymeric material and/or bound to at least one of the anterior surface and the posterior surface. In certain embodiments, the wound healing modulator can be a compound selected from the group consisting of antibiotics, antineoplastics, antimitotics, antimetabolics, anti-inflammatories, immunosupressants, and antifungals. The wound healing modulator compound can be selected from the group consisting of fluorouracil, mitomycin C, paclitaxel, ibuprofen, naproxen, flurbiprofen, carprofen, suprofen, ketoprofen, and cyclosporins. Further details of materials and additives are set forth in U.S. application Ser. No. 11/404,048, filed Apr. 13, 2006.

The opaque materials described above can absorb all or a substantial portion of the light that would otherwise pass through the ICR 47. Accordingly, the ICR 47 can comprise relatively rigid (e.g., metals, relatively hard materials, including hard plastics) or compliant materials. A compliant material can be a soft polymer material typically used to form contact lenses, such as flexible disposable contact lenses. A combination of rigid materials, compliant materials, and other types of materials can be used achieve the desired physical properties of the ICR 47.

Based on the configuration of the ICR 47, the light blocking material can be placed such that optical distortions are minimized. In some of the embodiments described herein, light blocking material forms at least portions of the peripheries of the ICRs that may otherwise cause some optical distortions. The inner periphery, outer periphery, or the entire ICR can be formed of an opaque material to improve eye functioning, although other portions of the ICR can comprise opaque material as well. The ICR 47 of FIGS. 6 and 7 comprises light blocking material.

In some embodiments, as shown in FIGS. 6A-B and 7, the ICR 47 has an inner periphery 140, a central portion 142, and an outer periphery 144 that cooperate to define a generally trapezoidal cross-section. The central portion 142 extends between the inner and outer peripheries 140, 144. The inner periphery 140 defines an inner edge 150 forming the aperture 110, and the outer periphery 144 defines an outer edge 154. At least one of the inner and outer peripheries 140, 144 is a light-blocking portion. In some embodiments, both of the inner and outer peripheries 140, 144 are light blocking portions that extend inwardly from a location near or at the inner and outer edges 150, 154. The illustrated peripheries 140, 144 comprise an opaque material and the central portion 142 comprises a somewhat transparent or optically clear material. In some embodiments, the central portion 142 also comprises an opaque material.

The inner and/or outer opaque peripheries 140, 144 can extend about at least a portion of the circumference of the elongated member 100. The illustrated inner and/or outer opaque peripheries 140, 144 extend about a substantial distance of the circumference of the elongated member 100. In some embodiments, the inner and/or outer opaque peripheries 140, 144 can subtend an angle of about 180 degrees, 270 degrees, 310 degrees, 340 degrees, 360 degrees, or any other suitable angle to reduce optical distortions when the ICR 47 is implanted as desired.

The illustrated opaque peripheries 140, 144 extend along the entire length of the elongated member 100. The widths of the outer peripheries 140, 144 can be selected so as to minimize edge effects due to light passing through the inner and outer front surfaces 160, 164, which are angled to the rear surface 162. As such, light that would otherwise scatter or otherwise cause visible distortions (e.g., edge effects) is blocked by the opaque material, thus improving the image viewed by the person's eye. The relatively thin opaque peripheries 140, 144 may not be visible to the patient, or at least do not appreciably obstruct viewing of the eye. As discussed above, the brain can disregard the nontransmissive portions of the ICR 47 to the extent these portions are visible.

The central portion 142 can comprise transparent material that is less opaque than the outer peripheries 140, 144. For example, the central portion 142 can comprise a non-opaque material, preferably a material that is generally optically clear, as noted above. Light can pass through the central portion 142. The inner and outer peripheries 140, 144 block a sufficient amount of light such that noticeable optical distortions are minimized, reduced, or completely eliminated. Accordingly, light passes through only specific portions of the ICR.

In some embodiments, the opaque material extends entirely between the inner and outer edges 150, 154 such that little or substantially no light passes through the ICR 47. Optical problems associated with light passing through the ICR 47 are therefore reduced or substantially eliminated. The dimensions of the ICR 47 can be small enough that the ICR 47 is cosmetically acceptable, and may not noticeably effect the person's viewed imaged.

With reference again to FIG. 6A, a width W is defined between the inner and outer edges 150, 154. A thickness T (FIG. 7) is defined by a front surface 142 and the rear surface 162. In some variations, the ratio of the width W to the thickness T can be 0.1 or more. In some variations, the ratio of the width W to the thickness T can be 0.2 or more. In some variations, the ratio of the width W to the thickness T can be 0.5 or more. In some variations, the ratio of the width W to the thickness T can be 0.5 or more. Because the ICR 47 is placed relatively close to the optical axis, the relatively thin ICR 47 can produce substantial changes to the curvature of the anterior surface 31 of the cornea 12 in the optical zone. In particular embodiments, the width W can be between about 1 micron and about 1000 micron and the thickness can be between about 1 micron and about 500 micron. The relatively flat ICR 47 can produce substantial curvature changes when the dimensions (e.g., the outer diameter) of the ICR 47 are sufficiently small. Compared to other shape change devices that are positioned well outside the optic zone, the ICR 47 is relatively thin.

In some non-limiting exemplary embodiments, the ratio of the width W to the thickness T is in the range of about 0.1 to about 10. In such embodiments, the ICR 47 can effectively change the curvature of the anterior surface 31 and still have a relatively slender configuration for minimal trauma to the cornea 12. In other embodiments, the ratio of the width W to the thickness T is in the range of about 0.2 to about 5. The ICR 47 can produce large corneal curvature changes for flexibility in refractive corrections while also being relatively easy to implant, and may reduce healing time. The width to thickness ratio of the ICR 47 can be selected based on the desired refraction correction, method of insertion, optical properties of the ICR 47, healing time, and the like.

The illustrated ICR 47 has a generally uniform width W and thickness T. In alternative embodiments, the ICR 47 can have a non-uniform width W and/or thickness T to correct, for example, astigmatisms, myopia, or other eye disorders. The ICR 47 can have a geometry that can be modified to correct for astigmatism. Such a device can include suitable features of astigmatic devices disclosed in U.S. Pat. No. 5,405,384, which is incorporated by reference. The ICR 47 can have disposed about its circumference or periphery thickened portions, widened portions, sloped sections, added bulk, and the like for flexibility to achieve the desired corneal correction.

In some embodiments, the ICR 47 can have one or more ribs on the posterior side, extending from the inner edge 150 to the outer edge 154. In some embodiments the rib(s) may extend substantially from the inner edge 150 to the outer edge 154 of the ICR 47. In alternative embodiments, the rib(s) may extend partially between the inner 150 and outer 154 edges of the ICR 47. The one or more ribs may provide additional structural support for the ICR 47. The one or more ribs can also be positioned to produce an additional change in the curvature of the cornea. In some embodiments, the one or more ribs can be used in conjunction with the shape and or thickness of the ICR 47 to produce the desired shape change in the cornea. In alternative embodiments, the one or more ribs may be positioned around the ICR 47 to provide a majority of the shape change to the cornea. The number and position of the rib(s) can be selected based on the desired refraction correction and/or the structural support needed for the ICR 47.

The inner diameter D₁ can be equal to or greater than about 1.0 mm, 1.5 mm, 2.0 mm, 3.0, 3.5 mm, 4.0 mm, and 4.5 mm, and ranges encompassing such lengths. In some embodiments, the ICR 47 defines an inner diameter D₁ of between about 2.0 mm and about 4.0 mm. In some embodiments, the outer diameter D₂ can be equal to or less than about 5.0 mm. In some embodiments, the outer diameter D₂ can be equal to or less than 4.5 mm. In some embodiments, the outer diameter D₂ can be equal to or less than 4.0 mm. In some embodiments, the outer diameter D₂ can be equal to or less than 3.5 mm. In some embodiments, the outer diameter D₂ can be equal to or less than 3.0 mm. In some embodiments, the outer diameter D₂ is equal to or less than about 2.5 mm, preferably between about 2.5 mm and about 4.5 mm. In these embodiments, the ICR 47 can produce large corneal changes while also being relatively small for easy implantation. In yet additional embodiments, the ICR 47 comprises an outer diameter D₂ in the range of about 2.5 mm to about 4.5 mm, and the inner edge 150 defines an inner diameter D₁ in the range of about 2.0 mm to about 4.0 mm. The patient's pupil, cornea, optical zone, and other eye components can be measured to determine an appropriately sized ICR for implantation. If a person's pupil has a diameter of 7 mm, the ICR 47 can have an outer diameter D₂ less than 7 mm. Optionally, the outer diameter D₂ can also be less than the diameter of the pupil when the pupil is constricted.

FIG. 6B illustrates another embodiment of an ICR 47′ that is similar to the ICR 47. The ICR 47′ is configured in one embodiment as a continuous ring. The ICR 47′ can have any suitable shape, such as being symmetrical about at least one axis. Single axis symmetry can be found in oblong, oval, egg shaped, and rectangular shaped devices. In one embodiment, the ICR 47′ is symmetrical about two axes. In the illustrated embodiment, the ICR 47′ is circular. The ICR 47′ can be configured with an enclosed circle at least one of an inner periphery 140′ and an outer periphery 144′. The illustrated embodiment of the ICR 47′ is of an unbroken continuous ring. In some embodiments, the ICR 47′ is separable at one or more locations, which can be seams in the device. As such, the device can be an elongate member that can be joined to provide a continuous ring structure. By providing a continuous ring structure, the ICR 47′ can be made more rigid than a structure having one or more gaps as disclosed herein. A more rigid structure can be advantageous in being easier to handle. Also, a more rigid structure can be useful in creating more shape change on a surface of the cornea, e.g., on an anterior surface of a cornea. Additionally, a continuous structure can more evenly affect the shape change of the patient's eye than where gaps of significant size are provided between first and second ends of an elongate member (as in FIGS. 6A and 7) or between separate inserts as discussed further below. Further details of a continuous ring structure are set forth in U.S. Pat. No. 6,280,470, which is hereby incorporated by reference herein in its entirety.

The ICR can be modified to comprise a plurality of ring segments that can cooperate to form a generally circular annulus, as shown in FIG. 8. The ICR 47a of FIG. 8 has a pair of arcuate ring segments 200, 202 each subtending an angle of about 180 degrees. The ends 210, 212 of the segment 200 are preferably near or coupled to the ends 214, 216, respectively, of the segment 202 when the ICR 47a is implanted. The ring segments 200, 202 can comprise opaque or transparent materials similar to the ICR 47 described above and can have similar or different cross-sections, widths, thicknesses, or other dimensions as the ICR 47 described above.

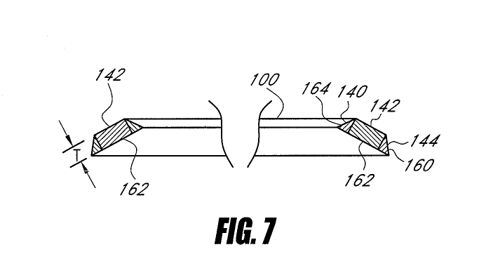

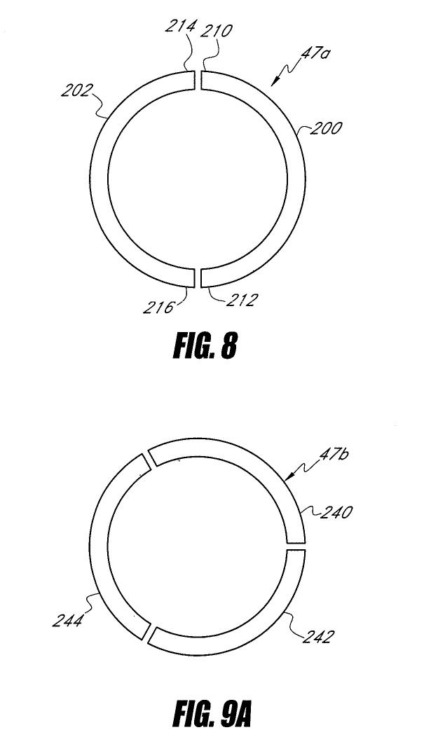

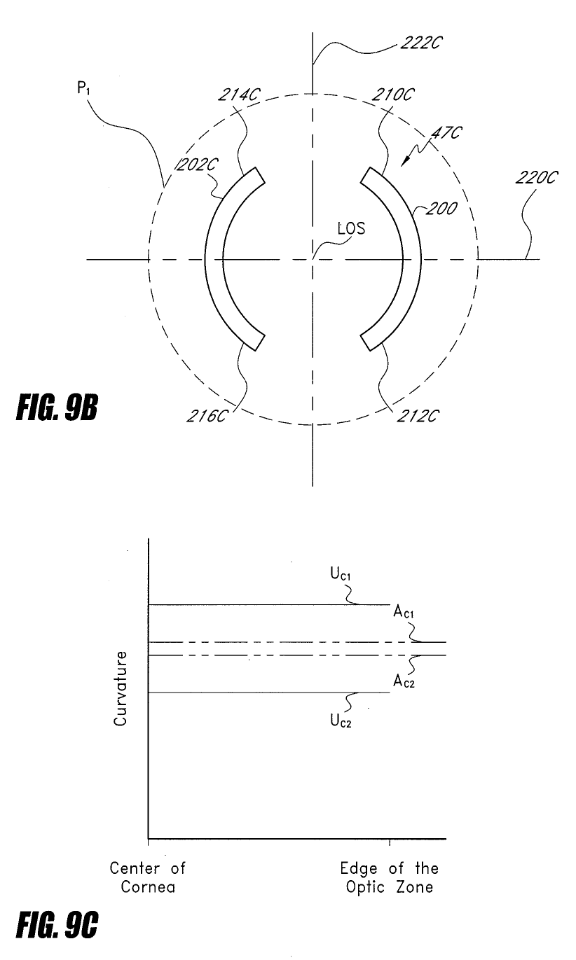

The ICR can be modified to comprise any suitable number of segments of any desired length. In some embodiments, including the illustrated embodiment of FIG. 9A, the ICR 47b comprises three ring segments 240, 242, 244 each subtending an angle of about 120 degrees. The curvature and size of the segments can be selected based on the number of segments and corrective refractive procedure to be performed. The segments of the ICR can have a similar shape and configuration as those disclosed in U.S. Pat. No. 5,824,086 entitled “Segmented Pre-Formed Intrastromal Corneal Inert,” which is incorporated by reference. In some embodiments, the ICR can have one or more ribs extending from the inner edge to the outer edge on the posterior side of each ring segment. The one or more ribs may provide additional structural support for the ring segments. The one or more ribs can also be positioned to provide an additional change in the curvature of the cornea. In some embodiments, the one or more ribs can be used in conjunction with the shape and or thickness of the ring segments to produce the desired shape change in the cornea. In alternative embodiments, the one or more ribs may be positioned around the ring segments to provide a majority of the shape change to the cornea.

Different techniques can be employed to implant the ICRs disclosed herein. For example, one or more flaps, pockets, channels, recesses, or other suitable structures can be formed in the cornea so that an ICR can be placed into the cornea. To implant the multi-piece ICRs, such as those of FIGS. 8 and 9, at least one incision can be formed in the cornea 12 so that a first arcuate segment can be inserted between a first corneal layer and a second corneal layer, preferably implanting the arcuate segment at a location anterior to a pupil of the eye. A second arcuate segment can also be inserted through the same incision and between the first corneal layer and the second corneal layer. Additional segments can be placed between the layers of the cornea 12 via a single incision. Alternatively, multiple incisions can be made in the cornea for implantation of the segments. In some methods of implantation, a separate incision can be formed to implant a single segment. For example, three incisions or pockets can be made to implant the three segments 240, 242, 244 illustrated in FIG. 9A.

In some methods of implanting, an intracorneal segment, such as those illustrated in FIGS. 8 and 9A, is installed in the following manner. A small radial incision is made at the corneal position (e.g., the corneal radius) at which the intracorneal segment is to be installed in the cornea. A dissector in the form of a split ring having a point suitable for producing the interlamellar channel in the corneal stroma is introduced into the stromal space through the small incision. It is then rotated in such a fashion that a generally semicircular or arc-shaped channel is formed partially circling the cornea at the chosen radius. The dissector is then rotated in the opposite direction to withdraw it from the tunnel or channel thus formed. The intracorneal segment is then introduced into the channel. The process can be used to implant any desired number of segments.

The segmented ICRs of FIGS. 8 and 9 can be used for correcting an eye having a condition such as myopia, hyperopia, and/or astigmatism. The size and placement of one or more segments may then be chosen based on the desired correction. For instance, the selected segments might be two segmented inserts subtending an arc of about 30 degrees. After implantation of the segments, the vision of the eye might be measured to determine whether adjustment or further correction is needed. If there is insufficient correction, the segmented inserts may be withdrawn and differently sized segments can then be implanted. If an astigmatic aberration is introduced, the insert may be withdrawn (partially or completely) and trimmed prior to complete re-insertion.

One advantage of a segmented design, such as in FIGS. 8 and 9, is that different effects in different planes can be accomplished. For example, FIGS. 9B and 9C illustrate a differential change in curvature provided by a two-segment arrangement. In this arrangement, first and second ends 210c, 212c of a first segment 200c are spaced from first and second ends 214c, 216c of a second segment 202c. The first and second segments 200c, 202c can be implanted in a patient's eye near and in some cases within the optical zone (e.g., between a projection of the iris PI and the patient's liner of sight LOS). As discussed above, in connection with some embodiments, in some variations at least one of the inner and the outer periphery of at least one of the first and second segments 200c, 202c is nontransmissive to visible light. As discussed above, the nontransmissive edge(s) reduce visible distracting optical aberrations. FIG. 9C illustrates the effect on the corneal curvature of the segments 200c, 202c. In one application, a first curvature change can be affected in a first plane 220c extending transverse to the first and second segments 200c, 202c while a second curvature change can be affected in a second plane 222c perpendicular to the first plane 220c. In one arrangement, the first and second segments 200c, 202c are configured such that the curvature in the first plane 220c is greater than the curvature in the second plane 220c. The locations of the first and second planes 220c, 222c can be selected based on a condition of the patient, such as an astigmatic condition. This may affect the number, configuration, and placement of the segments. FIG. 9C illustrates the first and second curvature UC1, UC2, which are the uncorrected curvatures of an astigmatic eye. After implantation of the segments 200c, 202c the curvature of a first adjusted curvature AC1 is provided in the first plane 220c and a second adjusted curvature AC2 is provided in the second plane 222c. FIG. 9C illustrates that while the first and second curvatures UC1, UC2 were different, the first and second adjusted curvatures AC1, AC2 are corrected to be substantially similar. In the illustrated embodiment, UC1 is reduced and UC2 is increased such that AC1 is similar to AC2. In some cases, AC1 and AC2 can be identical. In some cases, both curvatures are increased or decreased, with one of the curvatures being increased or decreased by a greater amount than the other. In other cases, one of UC1 and UC2 is not altered or is altered only by a small amount while the other of UC1 and UC2 is increased or decreased by a significant amount.

FIGS. 10 to 14 illustrate various configurations of axial cross-sections suitable for the ICRs illustrated in FIGS. 2A, 2B, and 5-9. The materials and cross-sections discloses herein can be interchanged based on the eye disorder. Generally, the ICRs can have axial cross-sections that are polygonal (including rounded polygonal), curved, semi-circular, and combinations thereof. With reference to FIG. 10, the ICR has an elongated, hexagonal axial cross-section 250 and may comprise mostly or all opaque material. In the illustrated embodiment, the entire ICR comprises an opaque, or light blocking material.

FIG. 11 illustrates an axial cross-section of an elongated segment 260 wherein one side comprises a light blocking portion 266 and a light-transmitting portion 268. The ICR can be implanted such that the light-blocking portion 266 forms the inner or outer periphery when implanted in a cornea. Additional light blocking portions can be added if needed or desired.

FIG. 12 is a cross-sectional view of another elongated segment that is generally trapezoidal. The elongated segment 270 has a pair of opposing outer light blocking portions 273, 276 that comprise different materials. The light blocking portion 273 can have a different opacity than the light blocking portion 276. The central portion 272 comprises a material having a relatively low opacity. In some embodiments, the central portion 272 also comprises a light blocking portion.

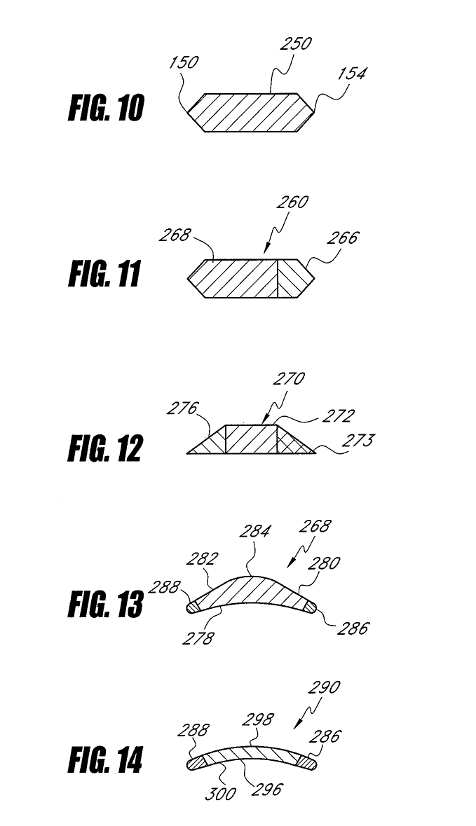

FIG. 13 is a cross-sectional view of another elongated segment 268 that has tapered outer portions 280, 282 extending outwardly from the thickened central portion 284. A bottom surface 278 of the elongated segment 268 can be generally curved so as to match the general curvature of a corneal layer. The illustrated bottom surface 278, for example, is somewhat concave and preferably matches the curvature of the lamella of the cornea in which the elongated segment 268 is implanted. As such, the elongated segment 268 can conform readily to the shape of the lamella so as to reduce or eliminate trauma to the cornea 12. The outer portions 280, 282 extend outwardly and terminate forming light blocking portions 286, 288. In some embodiments, the central portion 284 comprises a light blocking portion.

FIG. 14 is a cross-sectional view in accordance with another embodiment of an elongated segment 290 that is generally similar the elongated segment 268 illustrated in FIG. 13. The elongated member 290 has a generally uniform thickness defined between a convex upper surface 298 and a concave lower surface 300.

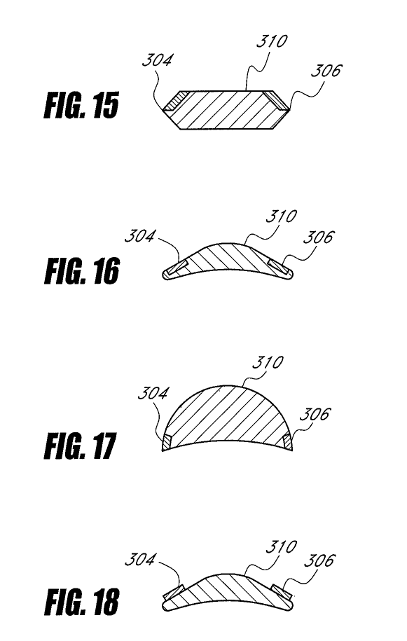

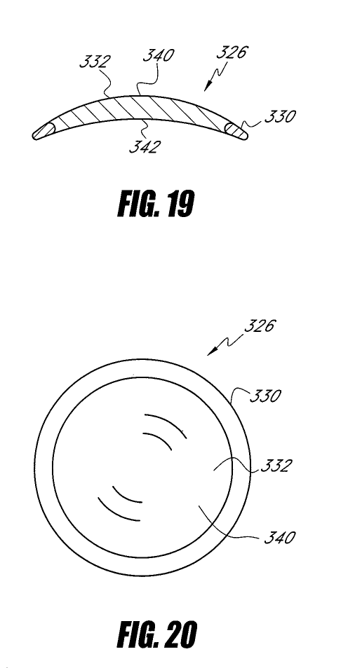

The ICRs illustrated in FIGS. 10-14 are illustrated as having light blocking portions that extend from an anterior (or front) surface to a posterior (or rear) surface. FIGS. 15-18 illustrated cross-sections of ICRs that comprise light blocking portions in the form of layers or coatings. The inwardly extending light blocking portions 304, 306 of FIGS. 15-18 can be formed by the coating, deposition processes, or other processes detailed above. The light blocking portions 304, 306 can be inlaid or inset as illustrated in FIGS. 15-17. In alternative embodiments, the light blocking portions 304, 306 can overlay an underlying central member 310, as illustrated in FIG. 18. The central portion 310 can be formed of a transparent material.

The ICR 47 can also have other cross-sections, such as those disclosed in U.S. Pat. Nos. 5,318,047, 5,405,384, 5,645,582, 5,653,752, 5,824,086, 5,944,752, 6,125,294, 6,214,044, each of which is incorporated by reference in its entirety. The patient's eye can be analyzed to determine an appropriate corrective procedure. The geometry (e.g., the cross-section) of the ICR can be selected based on the desired refractive correction.

The ICRs of FIGS. 2A, 2B, and 5-18 can be implanted after forming one or more channels or pockets in the cornea. A pocketing tool can be configured to form an intrastromal separation at a desired depth below the surface of the cornea. The pocketing tool can have a dissecting or delaminating tip configured to form the channel or pocket, such as the tools disclosed in U.S. Pat. No. 6,231,582 entitled “Corneal Pocketing Tool” which is incorporated by reference. Alternatively or additionally, the pocket tool can include an insertion stop feature positioned at a predetermined distance from the tip. As the tip of the pocketing tool is inserted vertically into the incision, a stop feature preferably contacts the outer surface of the cornea and effectively inhibits further advancement of the pocketing tool tip. With the depth of the delaminating tip controlled by the stop feature, the pocketing tool may then be manipulated to accurately create the desired separation.

The stop feature may also provide a steady reference against the surface of the cornea from which the pocketing tool may be rotated. Rotating the pocketing tool about such a reference provides an enhanced measure of control at the delaminating tip as it separates the lamella to form an initial separation or pocket. The pocketing tool is typically provided with an elongated handle positioned relative to the tip to provide the surgeon with optimum visual access and a steady grip and control as the instrument is manipulated.

The embodiments, features, systems, devices, materials, methods and techniques described in U.S. Pat. No. 5,846,256 entitled “Device and Method for Inserting a Biocompatible Material into the Corneal Stroma,” which is incorporated by reference, can be used to place ICR implants in the cornea as well. Channels connectors, finish channel connectors, probes, blades (e.g., dissector blades), and other instruments can be used to implant the ICRs described above. In some embodiments, a channel can be formed in corneal tissue for inserting an ICR into the corneal stroma of an eye. The method generally involves cutting at least one small incision into the corneal stroma. A blade, such as a clockwise or counter-clockwise dissector blade, can be inserted into the incision. The blade is then rotated clockwise or counter-clockwise to produce a clockwise or counter-clockwise channel. Another clockwise or counter-clockwise dissector blade is inserted into the incision and rotated clockwise or counter-clockwise to produce a clockwise or counter-clockwise channel. Additionally or alternatively, a probe is inserted clockwise into the clockwise channel and a counterclockwise probe into the counterclockwise channel to determine if the channels meet. Other types of pockets or channels can be formed by other methods as well. Centering guides and other devices disclosed in U.S. Pat. No. 5,403,335 entitled “Corneal Vacuum Centering Guide and Dissector,” which is incorporated by reference, can be used to implant and ICR.

The features, systems, devices, materials, methods and techniques disclosed herein may, in some embodiments, be similar to any one or more of the embodiments, features, systems, devices, materials, methods and techniques U.S. Pat. No. 5,188,125 entitled “Method for Corneal Curvature Adjustment,” U.S. Pat. No. 5,312,424 entitled “Corneal Curvature Adjustment Ring,” U.S. Pat. No. 5,403,335 entitled “Corneal Vacuum Centering Guide and Dissector,” U.S. Pat. No. 6,231,582 entitled “Corneal Pocketing Tool,” U.S. Pat. No. 5,964,776 entitled “Internal Keratome Apparatus and Method for using the Same to Form a Pocket/Flap Between Layers of a Live Cornea,” U.S. Pat. No. 5,964,748 entitled “Intrastromal Corneal Modification,” U.S. Pat. No. 6,083,236 entitled “Keratome Method and Apparatus,” U.S. Pat. No. 6,599,305 entitled “Intracorneal Lens Placement Method and Apparatus,” U.S. Pat. No. 6,623,497 entitled “Keratome Without Applanator,” U.S. Patent Application Publication No. 2004/0059361 entitled “Keratome,” U.S. Patent Application Publication No. 2005/0049621 entitled “Intracorneal Lens Placement Method and Apparatus,” which are all incorporated by reference in their entireties. The methods of implantation disclosed in these references can thus be used to form flaps, pockets, channels, etc. to implant the inserts disclosed herein.

In some embodiments, the implants described above can include a central lens. For example, the ICRs 47, 47′ of FIGS. 6 and 6A can surround a central lens that fills the aperture 110. The central lens can be a generally circular lens that can be coupled to the inner edge 150 of the ICR 47. In yet other embodiments, a central lens can be surrounded by the segmented portions illustrated in FIGS. 8 and 9. As used herein, the term “lens” is a broad term and includes, without limitation, concave, convex, bi-convex, bi-concave, generally planar, hemispherical, partially spherical, or other types of lenses suitable for implanting in the cornea. In the context of this application, any optical device that can change the refractive properties of the eye to improve vision can be a “lens” in this sense to be combined with any of the ICRs disclosed herein

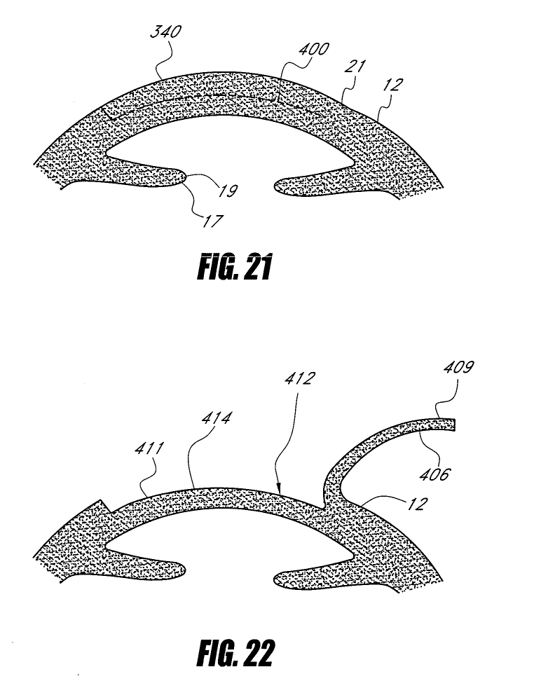

FIGS. 19 and 20 depict an intracorneal implant 326 including a light blocking portion 330 and a central portion 332. The intracorneal implant 326 can be adapted for implantation within the stroma of a cornea. As such, the intracorneal implant 326 can be an intrastromal implant in some applications. Other intracorneal devices disclosed herein can be similarly adapted and can be applied to the stroma using any suitable technique, such as those disclosed herein. The central portion 332 preferably is a lens made of an at least somewhat transparent material. As such, the intracorneal implant 326 can be placed in the optical zone of the eye. Light rays can pass through the central portion 332 but are blocked by the light blocking portion 330.

The illustrated light-blocking portion 330 is a generally annular ring that surrounds the circular central portion 332, as shown in FIG. 20. The light-blocking portion 330 can have a generally uniform width and can form the outermost portion of the implant 326. In alternative embodiments, the light blocking portion 330 has a variable width and/or may comprise a plurality of arcuate segments surrounding the central portion 332. The light-blocking portion 330 can be spaced from and separate from the central portion 332. Thus, the central portion 332 and light blocking portion 330 can form a system for correcting refractive errors that can be implanted together or separately in some applications. The size of the central portion 332 can be equal to or smaller than the inner size of any of the ICRs discussed above.

The central portion 332 can have a convex anterior surface 340 and a concave posterior surface 342. The central portion 332 can be used to adjust the curvature of the cornea as desired or can operate mainly by refraction at its surfaces to alter the refractive properties of the eye, as desired. For example, the central portion 332 can be made of a material selected to provide suitable refractive properties or can be made with a geometry selected to provide proper power (e.g., convex, bi-convex, positive or negative power meniscus, etc.). When implanted in the eye, the central portion 332 can provide corrective refraction, and the light-blocking portion 330 can reduce, minimize, or substantially prevent optical distortions while also providing corrective refraction.

FIGS. 21-24 illustrate a method of forming a flap and implanting the implant 326 of FIGS. 19 and 20. Analogous methods can be used to implant the ICRs illustrated in FIGS. 5-18.

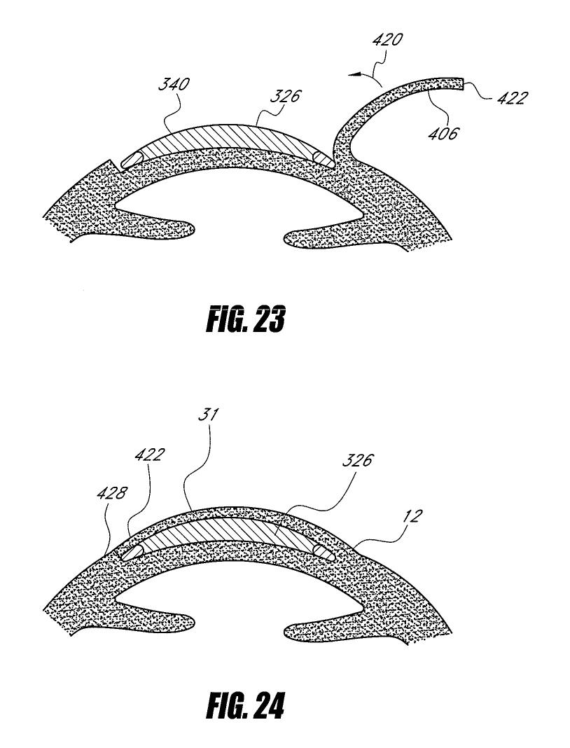

To form the flaps of FIG. 21, for example, a cutting tool (e.g., a blade, laser, femtosecond laser, etc.) can be used to form an incision 400 (shown in phantom) in the cornea 12. The incision is preferably generally parallel to the anterior surface 21 and between lamellar of the cornea 12. The illustrated incision 400 is positioned such that the first corneal layer 409 can be separated from a second corneal layer 411 at a location anterior to a pupil 19 of the eye, as shown in FIG. 22.

The flap 406 can be pulled away from the cornea 12 to expose an interior portion 412. The tougher outer layers of epithelial cells are separated and lifted away to expose the more compliant inner layers 414 of the cornea 12. The separated outer layers are left attached to the cornea 12 as flap 406 is pulled back. Once exposed, interior layers 414 of the cornea 12 may adjust themselves, or their shape may be altered through further surgical steps. Such further steps may include, for example, making radial keratotomy cuts or performing a subsequent resectioning which may include removing a contoured layer of corneal tissue.

Similar to the ICRs discussed above in connection to FIGS. 2A and 2B, the light blocking portions 330 of the insert 326 can have a smaller outer dimension than the inner diameter of the pupil 19 when the iris 17 is partially or fully dilated. In some embodiments, the insert 326 has an outer diameter between about 2.5 mm and about 4.5 mm. In other embodiments, the insert 326 has an outer diameter of between about 2.0 mm and about 4.0 mm.

As shown in FIG. 23, the implant 326 can be placed on the inner layers of the cornea 12. The flap 406 can be placed on the anterior surface 340 of the implant 326 until the flap 406 lays across the implant 326, as shown in FIG. 24. Preferably, the anterior surface 31 at the end 422 of the flap 406 is generally flush to the adjacent portion 428 of the anterior surface 31. The implant 326 can alter the refractive properties of the cornea by altering the curvature of the anterior surface 31 of the cornea 12 similar to the ICRs discussed above.

All patents and publications mentioned herein are hereby incorporated by reference in their entireties. Features and techniques of discussed in the following patents also may augment the disclosure herein, particularly for an ICR disposed within the optical zone of the eye. These patents are hereby incorporated by reference herein: U.S. Pat. No. 6,251,118 entitled “Radial Pocket Forming and Insert Positioning Instruments, Corneal Marker, and Method for using Same”; U.S. Pat. No. 6,231,582 entitled “Corneal Pocketing Tool”; U.S. Pat. No. 6,214,044 entitled “Hybrid Intrastromal Corneal Ring”; U.S. Pat. No. 6,175,754 entitled “Method and Apparatus for Measuring Corneal Incisions”; U.S. Pat. No. 6,051,023 entitled “Corneal Curvature Adjustment Ring and Apparatus for Making a Cornea”; U.S. Pat. No. 6,050,999 entitled “Corneal Implant Introducer and Method of Use”; U.S. Pat. No. 5,888,243 entitled “Hybrid Intrastromal Corneal Ring”; U.S. Pat. No. 5,846,256 entitled “Device and Method for Inserting a Biocompatible Material into the Corneal Stroma”; U.S. Pat. No. 5,824,086 entitled “Segmented Pre-formed Intrastromal Corneal Insert”; U.S. Pat. No. 5,785,651 entitled “Distance Measuring Confocal Microscope”; U.S. Pat. No. 5,766,171 entitled “Electrosurgical Procedure for the Treatment of the Cornea”; U.S. Pat. No. 5,693,092 entitled “Adjustable Devices for Corneal Curvature Adjustment”; U.S. Pat. No. 5,653,752 entitled “Adjustable Devices for Corneal Curvature Adjustment”; U.S. Pat. No. 5,645,582 entitled “Overlapping Ring Device for Corneal Curvature Adjustment”; U.S. Pat. No. 5,599,341 entitled “Laser Surgical Procedure and Device for Treatment of the Cornea”; U.S. Pat. No. 5,592,246 entitled “Device and Method for Mapping Objects”; U.S. Pat. No. 5,505,722 entitled “Corneal Curvature Adjusting Ring”; U.S. Pat. No. 5,475,452 entitled “Device and Method for Mapping Objects”; U.S. Pat. No. 5,466,260 entitled “Adjustable devices for corneal curvature adjustment”; U.S. Pat. No. 5,405,384 entitled “Astigmatic Correcting Intrastromal Corneal Ring”; U.S. Pat. No. 5,403,335 entitled “Corneal Vacuum Centering Guide and Dissector”; U.S. Pat. No. 5,323,788 entitled “Overlapping Split Ring Device for Corneal Curvature Adjustment”; U.S. Pat. No. 5,318,047 entitled “Method for Corneal Curvature Variation”; U.S. Pat. No. 5,312,424 entitled “Corneal Curvature Adjustment Ring”; U.S. Pat. No. 5,300,118 entitled “Adjustable Devices for Corneal Curvature Adjustment”; U.S. Pat. No. 5,188,125 entitled “Method for Corneal Curvature Adjustment”; U.S. Pat. No. 4,961,744 entitled “Holder for Inserting Corneal Curvature Adjustable Ring”; and U.S. Application Publication No. 2002-0010510 entitled “Variable Modulus Corneal Implant and Fabrication Methods.” In addition, the embodiments, features, systems, devices, materials, methods and techniques described herein may, in certain embodiments, be applied to or used in connection with any one or more of the embodiments, features, systems, devices, materials, methods and techniques disclosed in the above-mentioned incorporated disclosures.

The implants and ICRs disclosed herein may be formed through any suitable means. The various methods and techniques described above provide a number of ways to carry out the invention. Of course, it is to be understood that not necessarily all objectives or advantages described may be achieved in accordance with any particular embodiment described herein. Thus, for example, those skilled in the art will recognize that the methods may be performed in a manner that achieves or optimizes one advantage or group of advantages as taught herein without necessarily achieving other objectives or advantages as may be taught or suggested herein.

Furthermore, the skilled artisan will recognize the interchangeability of various features from different embodiments disclosed herein. Similarly, the various features and steps discussed above, as well as other known equivalents for each such feature or step, can be mixed and matched by one of ordinary skill in this art to perform methods in accordance with principles described herein. Additionally, the methods which are described and illustrated herein are not limited to the exact sequence of acts described, nor are they necessarily limited to the practice of all of the acts set forth. Other sequences of events or acts, or less than all of the events, or simultaneous occurrence of the events, may be utilized in practicing the embodiments of the invention.

Although the invention has been disclosed in the context of certain embodiments and examples, it will be understood by those skilled in the art that the invention extends beyond the specifically disclosed embodiments to other alternative embodiments and/or uses and obvious modifications and equivalents thereof. Accordingly, it is not intended that the invention be limited, except as by the appended claims.

What is claimed is:

1. An intracorneal implant for insertion into a cornea of a human eye, the eye having an anterior surface and a posterior surface, said implant comprising an elongated arcuate member comprising an opaque portion extending along at least a portion of a periphery of the arcuate member, and the implant being configured to alter the refractive properties of the cornea by altering the curvature of a surface of the cornea.

2. The implant of claim 1, wherein the implant is configured to alter the anterior surface of the cornea.

3. The implant of claim 1, wherein the implant is configured for placement in the cornea between the anterior surface and the posterior surface so that the implant surrounds an optical axis of the eye.

4. The implant of claim 1, wherein, when the opaque portion is exposed to incident light, the opaque portion blocks more than half of the incident light from passing therethrough.

5. The implant of claim 1, wherein the elongated arcuate member defines an inner diameter equal to or greater than about 2.0 mm.

6. The implant of claim 5, wherein the opaque portion extends from an outer edge of the elongated arcuate member.

7. The implant of claim 1, wherein the opaque portion comprises substantially all the elongated arcuate member.

8. The implant of claim 1, wherein the elongated arcuate member defines an inner diameter of between about 2.0 mm and about 4.0 mm.

9. The implant of claim 1, wherein the elongated arcuate member defines an outer diameter equal to or less than about 4.5 mm.

10. The implant of claim 1, wherein the elongated arcuate member defines an outer diameter of at least about 2.5 mm.

11. The implant of claim 1, wherein the elongated arcuate member defines an outer diameter of between about 2.5 mm and about 4.5 mm.

12. The implant of claim 1, wherein the elongated arcuate member comprises an outer edge and an inner edge, the outer edge defines an outer diameter in the range of about 2.5 mm to about 4.5 mm, and the inner edge defines an inner diameter in the range of about 2.0 mm to about 4.0 mm.

13. The implant of claim 1, wherein the distance between the anterior surface and the posterior surface defines a thickness, an inner edge and an outer edge of the elongated arcuate member defining a width, wherein a ratio of the width to the thickness is in the range of about 0.2 to about 5.

14. The implant of claim 1, wherein the elongated arcuate member is a circular annulus.

15. The implant of claim 1, wherein the elongated arcuate member comprises a first end, a second end, and an unbroken arc extending therebetween, the second end located adjacent to the first end in an implanted configuration.

16. The implant of claim 1, wherein the elongated arcuate member has an interior edge that at least partially forms a generally circular opening.

17. The implant of claim 1, wherein the implant comprises a substantially clear lens and the elongated arcuate member is disposed about a periphery of the lens.

18. The implant of claim 1, wherein the implant comprises a substantially clear lens and the elongated arcuate member forms at least a portion of a periphery of the lens.

19. The implant of claim 1, wherein implant further comprises another an elongated arcuate member comprising an opaque portion extending along at least a portion of a periphery of the arcuate member.

20. The implant of claim 1, wherein the elongated arcuate member comprises a plurality of segmented members configured to form a substantially arcuate implant when implanted.

21. A method for affecting the shape of a cornea of an eye, the method comprising:
separating a first corneal layer and a second corneal layer at a location anterior to a pupil of the eye; and
altering the anterior surface of the cornea to affect the refractive properties thereof by placing an opaque member between the first and second corneal layers.

22. The method of claim 21, wherein the opaque member comprises a first arcuate segment and a second arcuate segment, and altering the anterior surface further comprises placing the first arcuate segment between the first and second corneal layers on one side of a central portion of the pupil and placing the second arcuate segment between the first and second corneal layers such that the central portion of the pupil is between the first and second arcuate segments.

23. The method of claim 21, wherein the opaque member comprises a first end, a second end, and an unbroken arcuate segment extending therebetween, the method further comprising:
forming an incision in the anterior surface of the cornea;
forming an arcuate channel between the first and second corneal layers; and
inserting the first end, the arcuate segment, and the second end through the incision.

24. The method of claim 21, wherein the opaque member comprises a circular outer periphery comprising a diameter of between about 2.5 mm and about 4.5 mm.

25. The method of claim 24, wherein the opaque member comprises a circular inner periphery comprising a diameter of between about 2.0 mm and about 4.0 mm.

26. The method of claim 21, wherein a first normal projection of the opaque member onto a plane perpendicular to an axis that extends through a center of the pupil and through a geometric center of the eye is within a second normal projection of an inner edge of the iris of the eye onto the plane when the iris is dilated.

27. The method of claim 21, wherein an outer periphery of the opaque member defines a first dimension and an inner edge of an iris of the eye defines a second dimension when the pupil is dilated, and wherein the first dimension is less than the second dimension.
